(19) United States
(12) Patent Application Publication
(10) Pub. No.: US 2011/0265801 A1
(43) Pub. Date: Nov. 3, 2011

(54) Device for the Alleviation of Snoring and Sleep Apnoea

(76) Inventor: Stewart Cullen, Subiaco (AU)

(21) Appl. No.: 12/993,800

(22) PCT Filed: May 15, 2009
(86) PCT No.: PCT/AU2009/000615

§ 371 Date: Feb. 7, 2011

(30) Foreign Application Priority Data
May 21, 2008 (AU) 2008902517

(51) Int. Cl. A61F 5/56

(52) U.S. Cl. 128/848

(57) ABSTRACT

Apparatus for the alleviation of snoring and sleep apnoea comprising a face shield incorporating a cylinder, said cylinder containing a piston or diaphragm connected by a link to a tongue receptacle in which the distal end of the tongue of a subject is captured; said piston being outwardly displaceable within said cylinder by spring means, electrically-operated screw means, physically expansive means, shape-memory material means, or pneumatic means, all said displacement means incorporating rate regulating provisions, to draw said tongue receptacle and said tongue outwardly at a slow, controlled rate during sleep-induced relaxation, thereby reducing any tendency towards airway obstruction.

This invention relates generally to devices for the alleviation of snoring and sleep apnoea and, specifically, to such devices which function by displacing the tongue forwardly during sleep periods.

During sleep, it is common for airway obstruction to occur as a result of the apposition of the rear part of the tongue or soft palate with the posterior pharyngeal wall. A first result is snoring—the vibration of the oral tissues during respiration—which may be only a nuisance to a partner or perhaps result in sleep interruption. Exacerbated by obesity or loss of muscle tone in throat and oral tissues with aging, the condition may progress to obstructive sleep apnoea. This is a potentially lethal disorder in which breathing stops during sleep for 10 seconds or more for sometimes 300 times during a night of sleep. The condition has been associated with a variety of potentially serious medical conditions, such as hypertension, ischemic heart disease and stroke. It is thus desirable that diagnosis and treatment occur as early as possible.

Because of the ubiquity of the condition, a very wide variety of treatments has been proposed. These include surgery, positive ventilation methods and the use of many devices designed to provide mandibular advancement, forward displacement of the tongue or a combination of both. In the most common surgical method, uvulopalatopharyngoplasty, part of the soft palate is removed to prevent closure of the pharyngeal airway during sleep. The procedure is performed under general anaesthetic in the operating theatre. In a similar procedure, laser assisted uvulopalatoplasty, multiple treatments with laser light are employed to progressively scar and tighten the palate without removal of tissue. Therapists are now performing an improved procedure in which a single treatment with laser light is used to remove the uvula and trim the soft palate while the patient is sitting upright and fully awake. As uvulopalatopharyngoplasty may be painful and require a hospital stay of one to three days and a recuperative period of one to two weeks, it is attractive only to the most intractable sufferers. While uvulopalatoplasty is much easier to tolerate and may result in only a sore throat of one to two weeks duration and other minor effects, sufferers are still reluctant to undertake any irreversible treatment which may have unforseen consequences. Other treatments involving the insertion of bone anchors, as taught by Sohn et al in U.S. Pat. No. 5,988,171, or the implantation of magnets in the uvula, as taught by Nelson et al in U.S. Pat. No. 6,955,172 and the like are judged generally by sufferers to be quite unattractive.

Positive ventilation methods, of which the most common is continuous positive airway pressure (CPAP), involves the use of a mask to supply pressurized air to a sleeping sufferer. The air pressure required (also called the titrated air pressure) is determined by a sleep disorders physician during an overnight study in a sleep laboratory. The titrated air pressure is usually in the range 6 to 14 centimetres of water, but extreme cases may require as high as 25 to 30 centimetres of water. While the use of CPAP usually produces an immediate improvement in sleep quality and quality of life generally, the mask and hose to the CPAP machine are ugly and cumbersome and also generally quite unattractive to many sufferers.

It has long been known that control of tongue position may reduce the tendency for its rear part to relax against the posterior pharyngeal wall and, thereby, may alleviate snoring and, in many cases, sleep apnoea. Many disparate methods and apparatus have been developed, directed towards achievement of this end. An example of a simple device is that taught by Gibbons in U.S. Pat. No. 2,844,142. In this invention, a larger and smaller loop of a suitable flexible or elastic material are joined, the smaller loop passing around the tongue and the larger around the lower jaw. The invention is useful in any situation in which control of the tongue is required. In an invention taught by Walter in U.S. Pat. No. 5,666,973, a shell-like or conchiform device is supported in the mouth on the proximal end of a rigid coupling member, a concave surface of the device being shaped to conform to and receive the distal one third to one quarter of the tongue. A retaining element fixed to the distal end of the coupling element limits inward movement of the device. The inventor claims that the invention prevents the tongue from sliding back into a position in which it might obstruct the airway, but provides no proper explanation as to how the effect is achieved. In a method taught by Thomas in U.S. Pat. No. 5,988,170, a base plate is held against the face by a strap or elastic band and a rigidly flexible tongue depressing member extends into the mouth and presses downwardly against the tongue to urge it forwardly into a position abutting the lower teeth. In a method taught by De Voss in U.S. Pat. No. 6,467,484, a U-shaped fixation member is fixed to the upper row of teeth and a U-shaped brace or tongue catching member extends downwardly and rearwardly to exert pressure upon the tongue and maintain it in a forward position. In a method taught by Grosbois et at in U.S. Pat. No. 6,474,339, an arcuate, flexible intra-buccal-pharyngeal tube is employed to ensure the maintenance of a patent airway. The distal end of the tube is provided with suitable filtration means and, in use, is gripped in the teeth. In methods taught by Pivovarov in U.S. Pat. Nos. 6,675,804 and 7,137,393, a multi-lobed structure similar in arrangement and function to the shell-like or conchiform device taught by Walter is claimed to normalize night breathing. As with Walter, no proper explanation is provided as to how the effect is achieved. In a method taught by D'Agosto in U.S. Pat. No. 6,976,491, an anti-snoring appliance defining an airway is placed in the mouth and located by a flange bearing against the facial surface. The upper surface of the appliance is made convex and shaped to closely conform to the contours of the hard palate. The lower surface of the appliance is provided with a series of spaced, transverse, traction ridges which pull the tongue forward and prevent it from falling back to obstruct the airway. In a method taught by Tepper in U.S. Pat. No. 5,052,409, the tongue is passed between a resilient band and a hinged, narrow bridge of an appliance, the bridge being curved to match the contour of the tongue in the vicinity of its dorsum. The inventor claims that the tongue is able to position itself such that its anterior portion contacts the rugae of the palate to achieve the proprioceptive or tactile feel for proper tongue positioning. In this way, the tongue is encouraged to be positioned against the palate and the passage of air is redirected away from the mouth and the soft palate and through the nose. To stimulate regular swallowing, provision is made to incorporate into the appliance means to release a liquid stimulant in a controlled fashion. All of the inventions cited possess obvious defects or disabilities which might lead the casual observer to question their efficacy or utility.

In addition to the heterogeneous inventions described above, a class of inventions directed towards the control of snoring and sleep apnoea employs a common operating principle—the use of a receptacle to capture the distal zone of the tongue and, thereby, to positively extend and/or stabilize the tongue in a suitable position. In order to properly reduce the tendency for the rear part of the tongue to relax against the posterior pharyngeal wall, depending upon the particular subject, the distal end of the tongue must be extended to and stabilized in a position at least 20 to 40 millimetres forward of its normal fully forward position. For the purposes of the present invention, the normal fully forward position of the tongue is taken to be when the distal end of the tongue abuts the inner surface of the teeth or, as appropriate, the adjacent, posterior gum surface. An example of inventions falling into this class is the anti-snoring device taught by Samelson in U.S. Pat. Nos. 4,169,473 and 4,304,227. In this invention, an anti-snoring device comprises an integrally moulded body providing dental engaging portions and a rearwardly-opening central socket. The outer or distal portion of the user's tongue is held in the socket by negative pressure in a position level with or forwardly of the external facial surface. The device is located by a curved front plate bearing against the exterior surface of the lips and engagement of the upper and lower dental arches of dental engaging portions in the form of upper and lower U-shaped troughs. Another example is the tongue holding device taught by Wirt et al in U.S. Pat. No. 4,196,724 in which the tongue is wedgingly engaged with the rearwardly converging walls of a receptacle and maintained by suction. Suction is generated by a bellows or suction producing bulb and the distal end of the tongue is maintained within the mouth. A chin engaging element is provided to guide the proper positioning of the device. Another example is the device taught by Gardy in U.S. Pat. No. 4,676,240. In this invention, an integrally moulded body of pliable, elastomeric material incorporates a rearwardly opening central vacuum chamber. The tongue is held forwardly in the vacuum chamber with its distal part more or less level with the exterior facial surfaces. The device is located in the mouth by the teeth engaging restraining ridges formed on the exterior surfaces. Another example is the anti-snoring apparatus taught by Alvarez in U.S. Pat. No. 5,154,184. This invention comprises a receptacle structure configured for reception and retention of the outer extent of the user's tongue and a shield structure which is positionable on the receptacle structure. The shield structure abuts the exterior facial surfaces and its positioning on the receptacle structure determines the degree of extension of the user's tongue. The user's tongue may be stabilized within the mouth or with its distal part extending beyond the lips. Another example is the device taught by Forney in U.S. Pat. No. 5,373,859 which holds the tongue securely in a housing by means of vacuum created within the device. The vacuum is created either manually by the squeezing of a closed, flexible vacuum reservoir or by means of an electrically-operated control unit. The device is located within the mouth by an integral flange contacting the face and the distal part of the tongue is maintained more or less level with the exterior facial surface. Another example is the apparatus to position a user's tongue taught by Alvarez et al in U.S. Pat. No. 5,465,734. This invention comprises a tongue sleeve to position a user's tongue and bring it forward to reduce obstruction of the airway. The tongue is held in the sleeve by suction and the sleeve is located in the user's mouth by a shield restrained by the user's lips. The degree of extension of the user's tongue is regulated by adjusting the position of the shield on locating notches and ridges provided on the exterior of the sleeve. The tongue may be extended to and maintained in a position in which its distal end is beyond the lips. The sleeve is shaped such that the upper and lower teeth rest upon its sides when the mouth is closed, pressure of the teeth on the rearward portions of the sleeve tending to push it forward, thereby moving the tongue forward and achieving the desired effect. Another example is the device for treating obstructive sleep disorders taught by Kulick in U.S. Pat. No. 6,494,209. The device comprises a mouthpiece portion that fits within the anterior portion of the oral cavity much like the mouthpieces used in snorkel and scuba devices. The forward part of the mouthpiece protrudes outwardly through the lips of the user and includes a tongue-shaped cavity for supporting the tongue. Bite blocks are employed to maintain the teeth in spaced-apart relationship and prevent damage to the tongue. Tubing is led from the outer end of the mouthpiece portion to a suction source to reduce the pressure within the tongue-shaped cavity to maintain the tongue in place. The tongue may be extended to and maintained in a position in which its distal end is beyond the lips. Another example is the tongue stabilizing device taught by Robertson et al in U.S. Pat. No. 7,073,506. This device is made in one piece from a resiliently flexible material. It comprises a hollow interior, a bulb portion connected to the hollow interior by a neck section of narrower diameter and positional locating tabs which engage the user's exterior facial surface or are positioned between the user's teeth and lips. The tongue is held in the hollow interior in a forwardly located position by a depression created by squeezing the bulb to expel air, placing the tongue in the hollow interior and releasing the bulb. The device appears to be intended to extend and maintain the tongue in a position in which its distal end is level with the lips. Some of the examples cited incorporate air passages which permit their use by mouth breathers and some are directed at the prevention of mouth breathing.

All of the examples cited in which a receptacle is employed to capture the distal zone of the tongue and, thereby, to positively extend and stabilize the tongue in a suitable position, suffer from one or more principal shortcomings. Those that extend and maintain the tongue only in its normal fully forward position are of doubtful efficacy, particularly in obese subjects or those with poor muscle tone in throat and oral tissues. A number are too large or bulky to be comfortably accommodated in the oral cavity and, as a result, inhibit sleep. A number induce a proper degree of tongue extension to alleviate snoring and sleep apnoea, but this position is unnatural and uncomfortable for the tongue and therefore also inhibits sleep. Some incorporate a bellows or bulb external to the mouth for the formation of a depression within the tongue holding receptacle. Should a subject sleep with his or her face to the pillow, pressure upon the bellows or bulb may destroy the depression and release the tongue. Some, such as that taught by Kulick, use a separately located device to generate the depression, with the possibility of the inadvertent tangling or disconnection of tubing during sleep. A number of inventors acknowledge that use of their inventions does occasion discomfort which may inhibit sleep and suggest habituation procedures.

The object of the present invention is to provide a self-contained device to displace and stabilize the tongue in a position up to 40 millimetres beyond its normal fully forward position, a device which, in operation, requires no external power source, a device in which the parts accommodated within the oral cavity are minimal in terms of bulk, and a device in which the tongue extension is slow, progressive and automatic to minimise discomfort in a user and provide minimal inhibition to sleep.

According to the present invention, a device is provided for the alleviation of snoring and sleep apnoea in which the tongue is captured in a simple receptacle of minimal bulk and slowly and progressively drawn outwardly to stabilize in a position approximately 20 to 40 millimetres beyond its normal fully forward position. The receptacle is located in the oral cavity by an integral shield of the device abutting the facial area around the mouth. The mechanism to effect extension of the tongue is fixed to the exterior surface of the shield, is activated by the user and unaffected by the user's sleeping posture. Bite blocks are provided to ensure that the teeth are maintained in a spaced-apart relationship during sleep to permit egress of the tongue and receptacle and to allow the user to more readily secure the device in place during use.

To provide a supplementary therapeutic effect, provision is optionally made to forwardly displace the uvula by fixing magnetic means to or implanting such means within it and attracting the magnetic means through the use of complementary magnetic means. Such forward displacement of the uvula is acknowledged to similarly alleviate the snoring and associated conditions referred to herein.

The various aspects of the present invention will be more readily understood by reference to the following description of preferred embodiments given in relation to the accompanying drawings in which:

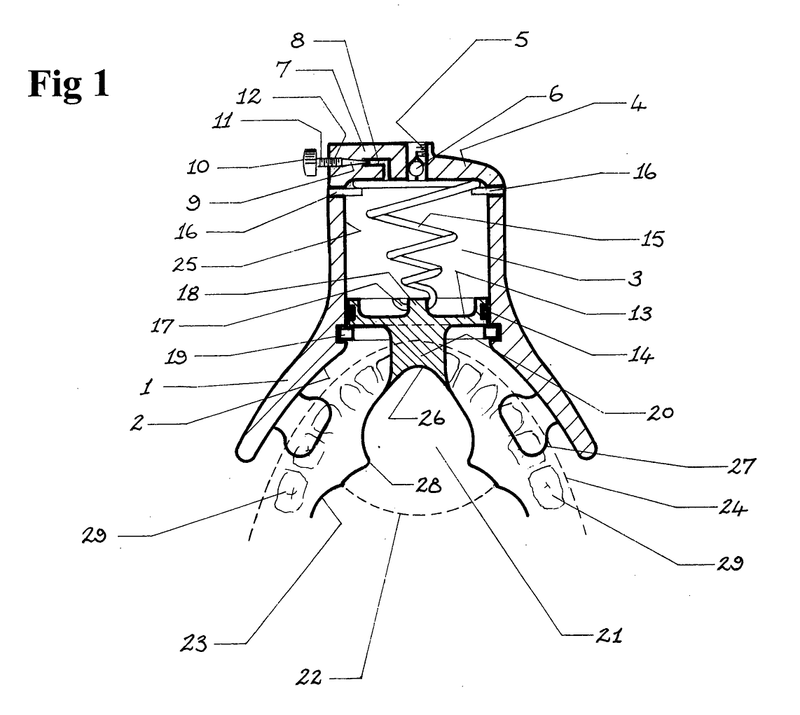

FIG. 1 is a partial longitudinal cross-sectional view of the present invention through a more or less horizontal plane;

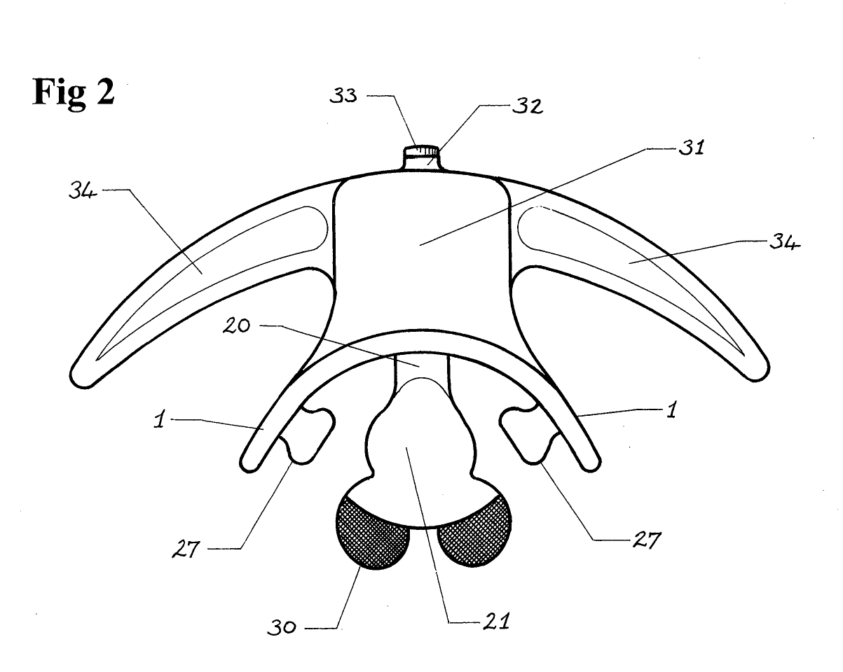

FIG. 2 is a view from above of a variation of the embodiment depicted at FIG. 1;

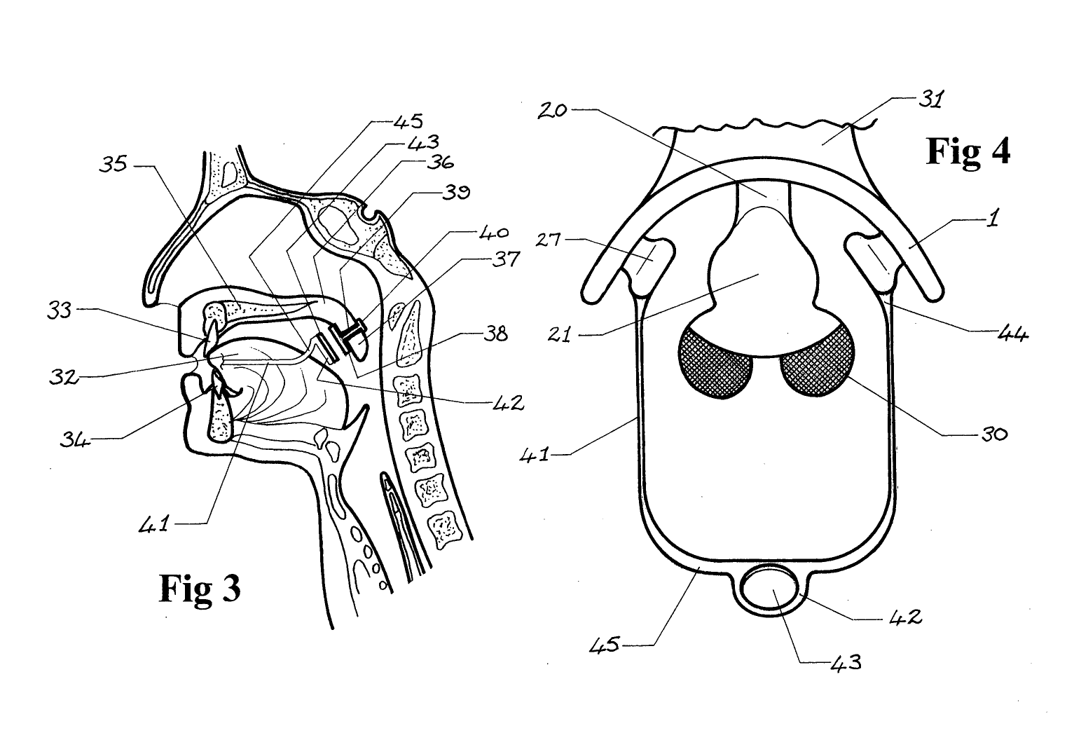

FIG. 3 is a partial cross-sectional view on a sagittal plane of the oropharyngeal region of the human head;

FIG. 4 is a partial view from above of a first alternative embodiment of the shield and tongue receptacle of the present invention;

FIG. 5 is a partial longitudinal cross-sectional view of a second alternative embodiment of the present invention through a more or less horizontal plane;

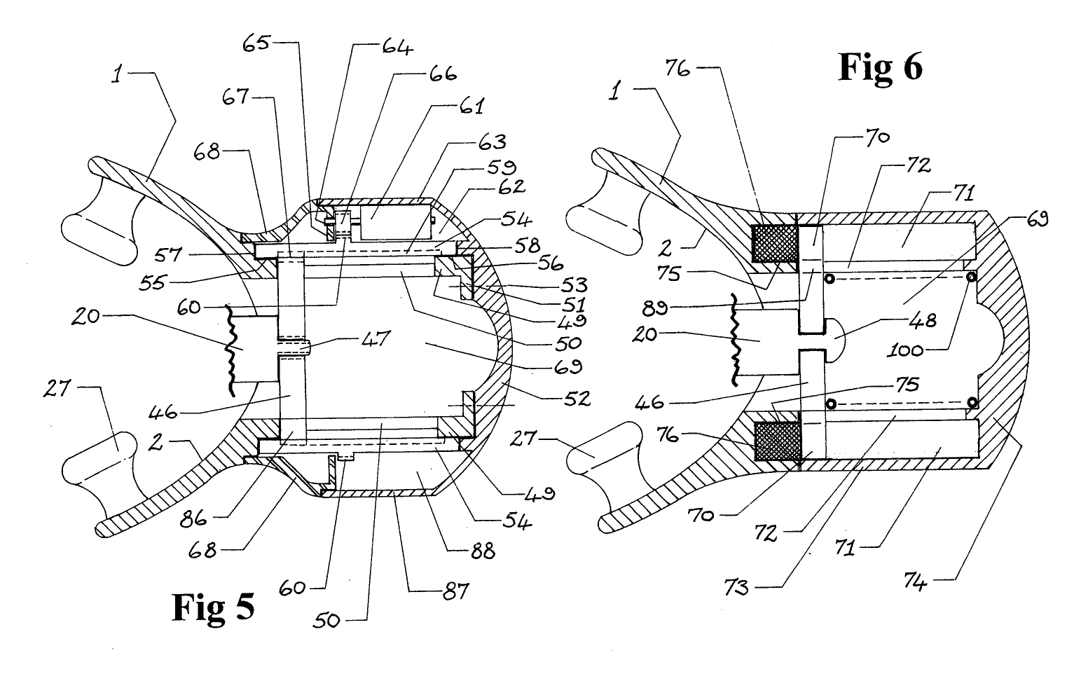

FIG. 6 is a partial longitudinal cross-sectional view of a third alternative embodiment of the present invention through a more or less horizontal plane;

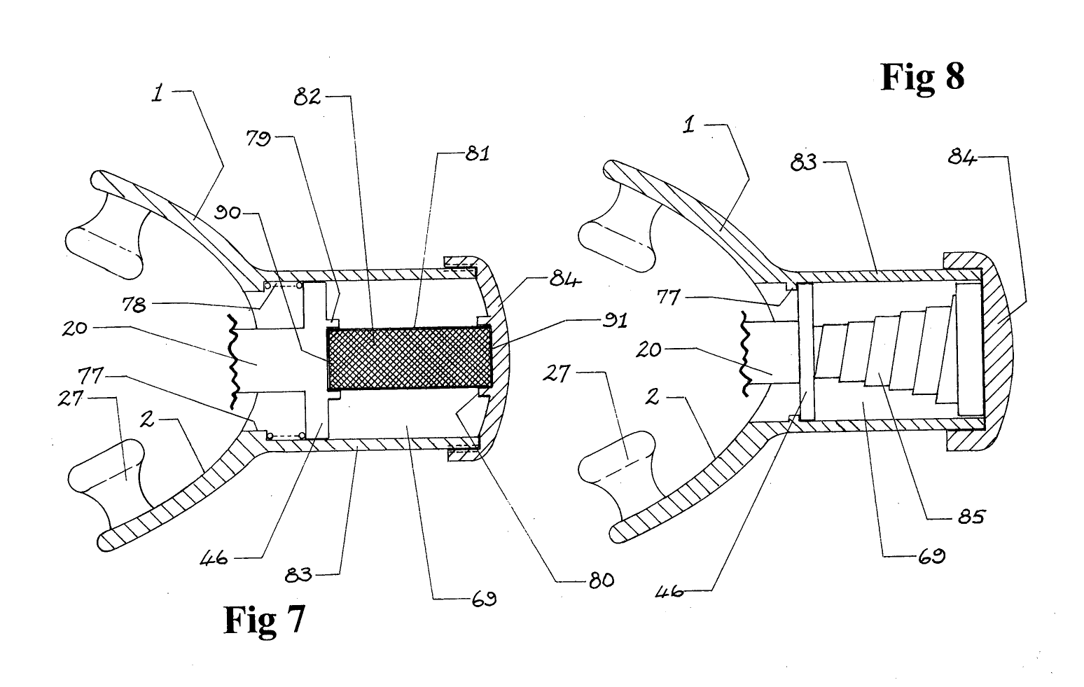

FIG. 7 is a partial longitudinal cross-sectional view of a fourth alternative embodiment of the present invention through a more or less horizontal plane;

FIG. 8 is a partial longitudinal cross-sectional view of a fifth alternative embodiment of the present invention through a more or less horizontal plane;

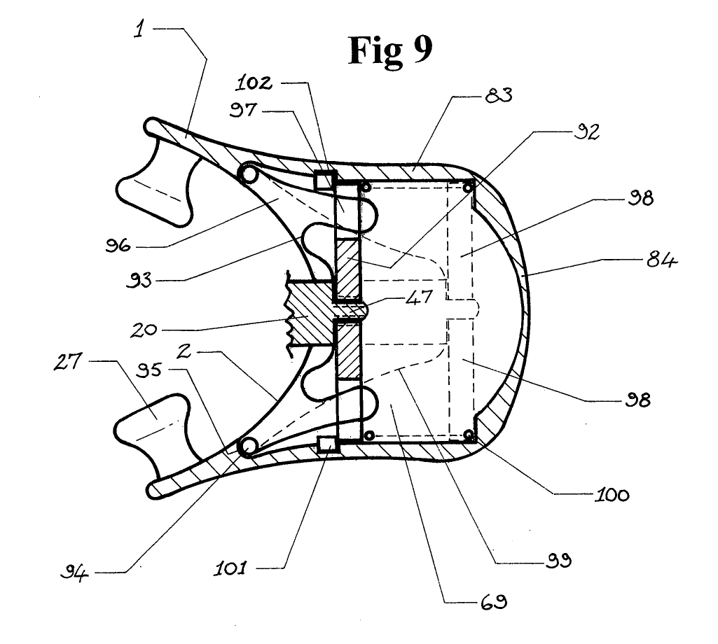

FIG. 9 is a partial longitudinal cross-sectional view of a sixth alternative embodiment of the present invention through a more or less horizontal plane;

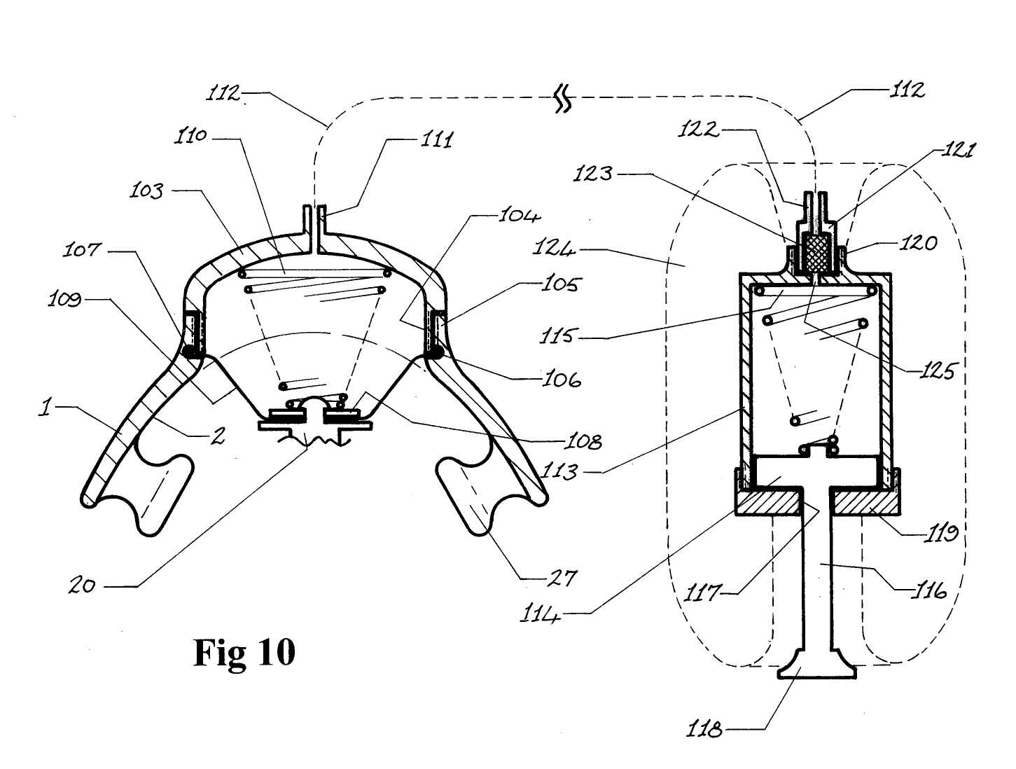

FIG. 10 is a partial longitudinal cross-sectional view of a seventh alternative embodiment of the present invention and its driving unit through a more or less horizontal plane.

It should be noted that the various embodiments of the present invention are depicted in the figures at differing scales.

With reference to FIG. 1, in a device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 3 is formed on the outer surface of said shield and is closed at its outer end by integral cap 4. Said cap incorporates threaded inlet fitting 5 in which is accommodated non-return valve 6 which admits an inflow of gas to said cylinder, but prevents any outflow. Valve block 7 is moulded onto said cap, said valve block incorporating bleed duct 8 closed by finely tapered needle valve 9. In the preferred embodiment, said needle valve engages a complementary hard metal seat formed in said bleed duct. Said needle valve is formed on the inner end of threaded 11 stem, on the outer end of which is formed knurled knob 10. Said threaded stem is screwed into threaded bore 12 and an anti-rattle spring is optionally provided between said knurled knob and the outer surface of said valve block. By appropriately turning said knurled knob, said needle valve is unseated from said hard metal seat and pressurized air from within said cylinder is able to escape via said bleed duct and the voids between said threaded stem and said threaded bore. In an alternative embodiment (not shown) threaded bore 12 is formed in a porous, sintered metal insert provided in said valve block and pressurized air from within said cylinder is able to escape via said bleed duct and the voids in said sintered metal insert. In another alternative embodiment, the outer end of said needle valve is made suitably shaped or slotted and positioned below the surface of said valve block, said needle valve being adjusted using a small screwdriver or other complementary tool.

Piston 13 is slidably accommodated in cylindrical bore 25 of said cylinder, suitable seals 14 acting to seal said piston to said bore. The axial depth of said piston is such as to provide sufficient bearing surface to ensure that said piston remains stable in said bore regardless of normal radial forces applied to link 20. Travel of said piston in said bore is limited by removable snap ring 17. Hook 17 provided on the smaller diameter end of tapered spring 15 is engaged with boss 18 formed on piston 13. The larger diameter end of said spring is captured at the inner surface of integral cap 4 by three or more radially arranged metal pins 16 sealing pressed into suitable bores in the walls of said cylinder immediately adjacent said integral cap. Said spring is a tension spring and acts to urge said piston towards said integral cap. Said spring is made tapered so that, when it is in its fully retracted condition, its coils lie in a more or less concentric, flat arrangement. In its free form, said spring is substantially inverted from the form depicted in the figure, such that, in its said fully retracted condition, it continues to urge said piston towards said integral cap. Link 20 connects the underside of said piston to tongue receptacle 21, the proximal edge 22 of which is indicated in broken line. Said tongue receptacle is made substantially flattened to conform to the shape of the tongue and a more or less circumferential constriction 28 is provided passing around all of its sides to promote sealing of the tongue to it. A user's tongue 23 is pushed into and worked in said tongue receptacle, thereby expelling air from said receptacle such that any attempt to withdraw said tongue is impeded by a depression existing between the distal end of said tongue and the inner distal end 26 of said tongue receptacle. The employment of such receptacles to receive and capture the outer zone of the tongue is well known in the art.

Suitably located bite blocks 27 are formed on the inner surface of shield 1. Said bite blocks are made sufficiently thick to ensure that the teeth (position shown ghosted as 29) are maintained in a spaced-apart relationship during sleep sufficient to permit egress of said tongue receptacle. To allow the user to more readily secure said device in place during use, in an alternative embodiment (not shown), a securing strap is connected to fittings formed on the rearward edges of said shield and passes around the user's head to better secure said device in place. The approximate inner surface of the upper lip is indicated in broken line as 24.

In use, a small air pump (not shown) provided with the said device is connected to threaded inlet fitting 5 and a prescribed amount of air pumped into cylinder 3. In the preferred embodiment, said pump is of the direct stroke type (bicycle pump-type). In alternative embodiments for persons having impaired dexterity of hand strength, the plunger of said pump is operated by the squeezing of two handles or by the turning of a suitable crank. In another alternative embodiment, a small electrically-powered air pump is employed to pressurize said cylinder. In the preferred embodiment, a pressure relief valve is incorporated into said pumps, any air pressure above a prescribed maximum being audibly released from said valve. Pressurizing of said cylinder acts to displace piston 13 to its proximal position in which, with the device in use, said tongue receptacle is located in the normal, relaxed position of the tongue. With said piston located in its proximal position, tapered spring 15 is fully extended, the force applied by said air pressure to said piston overcoming said urging of said spring. Said tongue receptacle is introduced into the oral cavity and the tongue firmly engaged with it in the manner described. Bite blocks 27 are gripped between the teeth to retain shield 1 in place against the face with said tongue receptacle properly located in the oral cavity. Prior to sleep, the user turns knurled knob 10 appropriately to just open needle valve 9, permitting the pressurized air within said cylinder to slowly bleeds away. It is expected that, with proper adjustment, some 20 or more minutes will elapse before sufficient pressure loss occurs for tongue extension to commence, during which period the user will have fallen asleep. As the air pressure within said cylinder falls, the urging of said tapered spring becomes dominant and said tongue receptacle (together with said tongue) is slowly drawn outwardly between the parted teeth and carried up into said cylinder behind said piston and into the desired position.

In the preferred embodiment, prior to pressurization of said cylinder, a small amount of a suitable non-toxic lubricant is inserted into threaded inlet fitting 5, said lubricant subsequently being carried into said cylinder with the pressurizing airflow and acting to lubricate the co-operating surfaces of said piston and said cylinder bore.

In an alternative embodiment (not shown), said tapered spring is deleted and a suitable cylindrical spring is provided between snap ring 19 and the underside of piston 13. In this embodiment, said cylindrical spring is coated with a suitable anti-friction material to eliminate the possibility of scoring of the bore 25 of cylinder 3.

In another alternative embodiment (not shown), said tongue receptacle is made detachable from link 20. In this way, tongue receptacles of different sizes and configurations may be incorporated into said device. In this embodiment, the proximal end of link 20 is made complementary with a fitting provided on the distal end of said tongue receptacle, the two said complementary parts being secured together with a suitable clip, pin or the like.

With reference to FIG. 2, said device for the alleviation of snoring and sleep apnoea is made with supporting bars 34 incorporated into or fixed to the external surfaces 31 of said cylinder. In the preferred embodiment, said bars curve backwardly to contact the cheeks of a user and act to prevent the imposition of excessive loads upon the face, gums and teeth of a user of said device while sleeping in a prone attitude. In the embodiment depicted, threaded inlet fitting 5 (as depicted in FIG. 1) is closed with a screwably inserted plug 33. In an alternative embodiment, said plug takes the form of a press-in dust cap. Also in the embodiment depicted, knob 10 (all as depicted in FIG. 1) has been deleted and threaded stem 11 shortened and set into threaded bore 12 where it is adjusted with a suitable tool. In this embodiment, tongue receptacle 21 is made with two inwardly projecting primary leaves 30 fixed to the inner edge of its lower panel. Said leaves are made from a suitable thin, soft, flexible material and are optionally formed as part of said tongue receptacle. Said leaves pass to either side of the lingual septum and are coated on their upper surfaces with a suitable metal material. In the preferred embodiment, said metal material is gold. Said primary leaves are each connected by means of a suitable single insulated conductor (not shown) to a suitable power source (not shown). Said power source is optionally made separate or is accommodated within a small compartment (not shown) formed on the external surface of said cylinder. Where said tongue receptacle is made removable, suitable plug-type connectors (not shown) are incorporated into link 20 and employed to complete the electrical connection during attachment of said tongue receptacle. In order to create an electrical circuit, small, secondary leaves (not shown) of similar material are also provided on shield 1 to make electrical contact with the inner lip surfaces and said secondary leaves are also to said power supply. In the preferred embodiment, the electrical current supplied by said power supply is direct current with the polarity such that stimulation occurs at said primary leaves. The voltage and current delivered by said power supply are such as to be just above the stimulus threshold and are optionally made adjustable to suit the electrical susceptibility of individuals. In an alternative embodiment, said electrical current is delivered by said power supply in pulsed form. Contact of said metal material with the underside of the tongue acts to stimulate the tongue towards an extended position and counteracts any tendency for the tongue to relax into a retracted position. The effect upon tongue position caused by metal contact with the underside of the tongue is well known in the art. The provision of said leaves serves to inhibit tongue retraction prior to the effecting of tongue extension in the manner described herein. In an alternative embodiment (not described), supporting bars 34 are provided towards their outer ends with flat pads of suitable shape and area which contact the cheeks of a user. Said pads are optionally padded and act to better distribute any forces against the face of a user. In another alternative embodiment (not shown), said supporting bars are deleted and replaced with a suitably shaped cheek plate which contacts a substantial part of the left and right cheek areas of a user. In the preferred embodiment, said cheek plate is specially moulded from an impression of the user's face.

With further reference to FIG. 1, in another alternative embodiment (not shown), non-return valve 6, valve block 7, bleed duct 8, spring 15, pins 16, needle valve, threaded stem and knob 9, 10, 11, and threaded bore 12 are deleted and the proximal end of a light, flexible air line having a very small bore is terminated at threaded inlet fitting 5. The distal end of said air line terminates at a small, light, compact evacuation unit which is set to gradually generate a depression of increasing magnitude over a predetermined time interval. Said depression is transmitted to piston 13 via said air line and acts to displace said piston and thereby to draw said tongue receptacle and the tongue towards the extended position in the manner described previously herein. In the preferred embodiment, said air line is sufficiently long to permit said evacuation unit to be positioned where it will not inhibit a user from getting to sleep. Also in the preferred embodiment, said evacuation unit takes the form of a piston in a cylinder which is progressively displaced by a small electric motor or other form of suitable actuator acting through screw or rack and pinion operating means. Provision is made for said operating means to be disconnected to permit their resetting prior to use. In the preferred embodiment, said electric motor or actuator is battery operated.

In another alternative embodiment (not shown), said air line is deleted and replaced by a light Bowden-type cable which passes from an actuator unit to piston 13. Said actuator unit acts to directly displace piston 13 via said Bowden-type cable. Similarly, said Bowden-type cable is made sufficiently long to permit said actuator unit to be positioned where it will not inhibit a user from getting to sleep. Said actuator unit incorporates a small electric motor or other form of suitable actuator which acts through screw or rack and pinion operating means to progressively displace said Bowden-type cable. Provision is made for said operating means to be disconnected to permit their resetting prior to use. In the preferred embodiment, said electric motor or actuator is battery operated.

With reference to FIG. 3, a partial cross-sectional view on a sagittal plane is shown of the oropharyngeal region of the human head, including a partial view of an alternative embodiment of said device for the alleviation of snoring and sleep apnoea. The view includes the soft palate 36 suspended from the posterior border of the hard palate 35 and the uvula 37 taking the form of a conical process formed on the lower medial edge of the soft palate. For the purposes of illustration, the tongue 32 is depicted displaced forwardly against the teeth 33, 34 in a position to minimise any tendency towards upper airway blockage. In order to minimise any tendency for the soft palate and uvula to be displaced inwardly and thereby cause upper airway blockage, one or more small, powerful magnets 38 are fixed to the anterior surface of the soft palate and the root of the uvula. In the preferred embodiment, said magnets are fixed to the anterior ends of short stems 39 which pass through suitable holes pierced in the soft palate and uvula, said stems being secured in place by means of suitable retainers 40 fixed to their posterior ends in abutment with the posterior surfaces of said soft palate and uvula. In the preferred embodiment, said retainers are removable and take the form of small, approximately circular plates clipped or screwed to the ends of said stems. Also in the preferred embodiment, said magnets, stems and retainers are, as appropriate, covered with or made from suitable biocompatible materials. In an alternative embodiment (not shown), said magnets are implanted wholly within said soft palate and uvula.

With additional reference to FIG. 4, in the first alternative embodiment of said device for the alleviation of snoring and sleep apnoea depicted in FIG. 3, the outer ends 44 of inwardly-extending support arms 41 are fixed to the lower, inner ends of bite blocks 27 formed on shield 1. Said support arms pass rearwardly inside the dental arch and along the lateral edges of the tongue in a position in which they offer no impediment to closure of the jaws on said bite blocks. Said support arms are joined at their inner ends by arch 45 which passes up and over the posterior part of the tongue. Fixed to the centre of said arch in a position just forward of said soft palate is magnet supporting plate 42 to which, in turn, are fixed one or more small, powerful magnets 43. In the preferred embodiment, magnets 38, 43 are of the neodymium, rare-earth type which are capable of generating sufficient attractive effort to effect and maintain the required degree of forward displacement of said soft palate and uvula. Said magnets are selected such that unlike poles are adjacent when said invention is placed properly in the mouth. In the preferred embodiment, said magnet supporting plate is angled in relation to said arch to support magnets 43 in a position more or less parallel to magnets 38. Said magnet supporting plate is shaped to accommodate an array of magnets complementary to an array of magnets fixed to or implanted in said soft palate and uvula.

In the preferred embodiment, said support arms are made from a suitable light, stiff material with a cross-sectional shape rectangular or more or less elliptical. The ratio of major to minor axes is in the range 3:1 to 8:1 and the major cross-sectional axis is orientated more or less vertically. To better accommodate the shaping of individual mouths, said support arms are optionally varied in length, varied in spacing, splayed inwardly or outwardly, angled upwardly or downwardly, or joggled upwardly or downwardly at some point along their length. Similarly, said arch is made to extend higher or lower and is tilted forwardly or rearwardly. In the preferred embodiment, said support arms are made removable, their outer ends 44 being accommodated in complementary channels (not shown) formed in the inner surfaces of said bite blocks and secured in place with small screws (not shown). In a first alternative embodiment (not shown), said outer ends of said support arms are made tapered and are frictionally captured in complementary sockets provided in said bite blocks. In a second alternative embodiment, said outer ends of said support arms are simply fixed to said bite blocks.

In use, with the mouth widely open, shield 1 is pushed firmly against the lips such that it is displaced slightly inwardly of its normal position, allowing the anterior zone of said tongue to be captured in said tongue receptacle and magnets 38, 43 to engage. Said shield is then displaced forwardly into its normal position and retained there by closing of the bite onto said bite blocks. In forwardly displacing said shield in the manner described, said magnets remain engaged, causing said soft palate and said uvula to be similarly displaced forwardly and retained in that position. The displaced position of said soft palate and said uvula are unaffected by the subsequent further forward displacement of said tongue and said tongue receptacle in accordance with the operating principles of the present invention.

In an alternative embodiment (not shown), said support arms, said arch and said magnet supporting plate are deleted and the central part of the upper, inner edge of said tongue receptacle is extended rearwardly in the form of a leaf having its free end adjacent the uvula. Said leaf is made from a suitable thin, stiffly flexible material and is shaped such that sits lightly upon the upper surface of the tongue in the normal, relaxed position of that organ. The inner end of said leaf is made strongly magnetic with an opposite polarity presented to said magnets fixed to or implanted in the uvula. In the preferred embodiment, said inner end of said leaf is made from or coated with a magnetic polymer material of the type well known in the art. In a second alternative embodiment (not shown), one or more small, powerful magnets of suitable polarity are fixed to the free end of said leaf. The magnetic material of said leaf is capable of generating sufficient attractive effort to effect and maintain the required degree of forward displacement of said soft palate and uvula.

With reference to FIG. 5, in a second alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 49, formed on the outer surface of said shield, is provided at its outer end with cap attachment flange 51. The open, outer end of said cylinder is closed by cap 52 fixed to said cap attachment flange by suitable fastenings (positions indicated as 53). Two or more narrow, axially-arranged slots 50 extend substantially throughout the length of said cylinder. Piston 46 is slideably accommodated within said cylinder, stiff, narrow blades 86 made complementary with said axially-arranged slots extending radially from said piston through said slots. Link 20 connected to tongue receptacle (not shown) is fixed to said piston by suitable connection means 47. In the preferred embodiment, said connection means take the form of bayonet-type, quick-connect/disconnect elements. Thread pads 67 formed on the outer ends of said narrow blades are provided on their outer surfaces with areas of threading which conform to and engage complementary threading 59 provided on the inner surface of threaded drum 54. Said threaded drum is rotationally supported in plain bearings 55, 56 formed on the exterior of said cylinder and is restrained from axial movement by its abutting thrust surfaces 57, 58 formed, respectively, in the base of said cylinder and in said cap. Said threaded drum is rotationally displaced on said plain bearings by circumferential rack 60 formed on its exterior surface being engaged by pinion 66 driven in rotation by small, powerful electric motor 61. Rotation of said threaded drum in the correct sense causes said thread pads, said blades, said piston and, thereby, said link and said tongue receptacle to be axially displaced outwardly in the manner desired. Cap 52 has formed on it at each side two axially-extending shells 63, 87 the edges of which abut the exterior surfaces of said cylinder and enclose, respectively, compartments 62, 88. The open, inner ends of said compartments are closed by moulding 68, the ends of which index accurately with the base of said cylinder and said compartment open ends. Said electric motor is accommodated within compartment 62, mounted to the inner surface of shell 63 with the outer end of its shaft 64 rotationally supported in a suitable bearing formed in web 65 of moulding 68. Accommodated within compartment 88 are a battery and control unit (not shown). Said battery is optionally made replaceable or rechargeable. Where said battery is made rechargeable, suitable miniature connection means are provided beneath a suitable removable or displaceable cover. Such miniature connection means and covers are commonly employed in small electronic devices and are well known in the art. Said control unit comprises on/off control, timing circuit, load sensing circuit and reset circuit. Said on/off control takes the form of miniature switch means which are optionally concealed beneath said charging connection cover or a similar cover, said switch means being actuated prior to commencement of use of the present invention. In operation, with said switch means actuated to the ‘ON’ position, said tongue receptacle installed in the proper way in the oral cavity of a user and with bite blocks 27 gripped between the user's teeth, said timing circuit delays operation of said electric motor by a pre-set time interval. In the preferred embodiment, said time interval is software-controlled and settable between zero and 60 minutes. Upon expiry of said time interval, said control unit actuates said electric motor such that said components are displaced outwardly and the user's tongue is drawn out to the desired position to alleviate said symptoms. In the preferred embodiment, said tongue displacement takes place over a time interval of between five and 20 minutes, said time interval being software-controlled. When said piston reaches the physical outer limit of travel, obstruction of said electric motor operation is detected by said control unit as a rise in current and said electric motor is immediately deactivated. To reset said piston, the user positions said switch means to the ‘OFF’ position, said reset circuit immediately powering said electric motor in the reverse sense and immediately deactivating it when a current rise is detected indicating that said piston has reached the physical inner limit of travel. Suitable conductors are provided between said control unit and said electric motor, optionally moulded into said cylinder or into said cap. Indexing means are provided between said cylinder, moulding 68 and said cap to ensure that said components are assembled in correct alignment.

In an alternative embodiment (not shown), chambers 62, 88 are extended circumferentially to meet, thereby creating a single annular chamber around said cylinder.

In another alternative embodiment (not shown), said electric motor and control unit are replaced with a clockwork actuation unit, the driving spring for said clockwork actuation unit being accommodated within an annular chamber formed around said cylinder.

With reference to FIG. 6, in a third alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 73 is made separate and is removably fixed to the outer surface of said shield with suitable attachment means (not shown). In the preferred embodiment, said attachment means take the form of multiple, bayonet-type, quick-connect/disconnect elements. Said shield is made thicker at its outer end to accommodate two or more, symmetrically-positioned, axial-arranged chambers 75 formed in it. Said cylinder is made with complementary, axially-arranged chambers 71 which extend substantially throughout its length. Said attachment means are arranged such that said cylinder can only be fixed to said shield with the axes of chambers 75, 71 in coincidence. Two or more narrow, axially-arranged slots 72 extend substantially throughout the length of said cylinder and connect the interior of said cylinder to chambers 71. Large piston 46 is slideably accommodated within said cylinder, stiff, narrow blades 89 made complementary with said axially-arranged slots extending radially from said large piston through said slots. Small pistons 70 are fixed to the outer ends of said blades and are slidingly accommodated within chambers 71. Extension spring 100 provided within cylinder 73 urges piston 46 towards its retracted, rearwards position. Link 20 connected to tongue receptacle (not shown) is fixed to said piston by suitable connection means 48. In the preferred embodiment, said connection means take the form of bayonet-type, quick-connect/disconnect elements. Expansion capsules 76 are made from a finely foamed, stiff elastomeric material. Between usages, said expansion capsules are placed at room temperature in chambers of a suitable press (not shown) in which pressure is applied to compress said capsules and reduce them to their minimum volume. Said press is then placed in refrigerated means to cool said capsules to a temperature in the range −5° C. to −10° C. Immediately prior to use of the present invention, said capsules are removed from said press, installed in chambers 75 and cylinder 73 fixed to said shield. Said tongue receptacle (not shown) is installed in the oral cavity, the user's tongue is captured in the manner described and the device gripped by closing the bite onto bite blocks 27. During sleep, said capsules are slowly warmed by body heat and resume their uncompressed shape, the force generated being sufficient to displace said small pistons outwardly in chambers 71 against the urging of extension spring 100 and thereby to similarly displace said blades, said large piston, said link and said tongue receptacle such that the user's tongue is drawn out to the desired position to alleviate said symptoms.

With reference to FIG. 7, in a fourth alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 83 is made integral with said shield and is closed at its outer end by removable cap 84. In the preferred embodiment, said cap is secured in place on the open end of said cylinder by means of multiple, bayonet-type, quick-connect/disconnect elements. Piston 46 is slideably accommodated within said cylinder, recess 90 being formed in the outer surface of said piston, optionally by the raising of a low enclosure 79. Link 20 connected to tongue receptacle (not shown) is made integral with said piston. In an alternative embodiment (not shown), said link is fixed to said piston by suitable connection means in the form of bayonet-type, quick-connect/disconnect elements. Piston spring 78 is positioned between shoulder 77 at the inner end of said cylinder and said piston, the force of said spring being sufficient to outwardly displace said piston, said tongue receptacle and the relaxed tongue of a user. Complementary recess 91 is formed in the inner surface of said cap, optionally by the raising of a low enclosure 80. Capsule 82 comprises a thin closed tube of a suitable elastomeric material filled with ice or a gel and maintained at a suitable temperature to maintain its elongated, cylindrical form. Prior to use of the present invention, said cap is removed from said cylinder, a said capsule is placed in said cylinder with its ends accommodated in recesses 90, 91 and said cap replaced. During said installation of said capsule, said piston is displaced inwardly in said cylinder against the urging of piston spring 78. In use, said tongue receptacle (not shown) is installed in the oral cavity, the user's tongue is captured in the manner described and the device gripped by closing the bite onto bite blocks 27. As the body heat of the user raises the temperature of said capsule, said ice or said gel reverts to liquid form, permitting said piston to be displaced outwardly in said cylinder together with said link and said tongue receptacle such that the user's tongue is drawn out to the desired position to alleviate said symptoms.

With reference to FIG. 8, in a fifth alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 83 is made integral with said shield and is closed at its outer end by removable cap 84. In the preferred embodiment, said cap is secured in place on the open end of said cylinder by means of multiple, bayonet-type, quick-connect/disconnect elements. Piston 46 is slideably accommodated within said cylinder and link 20 connected to tongue receptacle (not shown) is made integral with said piston. In an alternative embodiment (not shown), said link is fixed to said piston by suitable connection means in the form of bayonet-type, quick-connect/disconnect elements. Shoulder 77 at the inner end of said cylinder acts to limit the inward displacement of said piston. A suitable spring 85 made from a suitable shape memory polymer material or shape memory metal alloy material is detachably fixed to the outer surface of said piston and permanently fixed to the inner surface of said cap. Said shape memory polymer material or shape memory metal alloy material has a transition temperature close to or slightly below normal human body temperature. The undistorted form of said spring is a flat disk with all coils immediately concentric and the force generated by said spring in reverting to its undistorted form is sufficient to outwardly displace said piston, said tongue receptacle and the relaxed tongue of a user. Between usages, said cap is removed and placed in a suitable press (not shown) to fully extend said spring. Said press is then placed in refrigerated means to cool said spring substantially below said transition temperature. Immediately prior to use, said cap is installed on the open end of said cylinder, said piston being displaced inwardly in said cylinder to abut shoulder 77. The inner end of said spring is provided with suitable connection means to fix it to the outer surface of said piston. In the preferred embodiment, said connection means take the form of bayonet-type, quick-connect/disconnect elements which are engaged or disengaged as said cap is installed or removed. In use, said tongue receptacle (not shown) is installed in the oral cavity, the user's tongue is captured in the manner described and the device gripped by closing the bite onto bite blocks 27. As the body heat of the user raises the temperature of said spring, it reverts to its undistorted form, displacing said piston outwardly in said cylinder together with said link and said tongue receptacle such that the user's tongue is drawn out to the desired position to alleviate said symptoms.

In an alternative embodiment (not shown), a suitable air passage is provided through said link, said piston and said spring to exhaust via a suitable exhalation valve. Said exhalation valve permits an outflow of air only and, in the preferred embodiment, is made from a thin, light elastomeric material and of the type commonly used in diving equipment and well known in the art. During respiration, at least some expired air passes out through said spring and serves to raise its temperature to body heat.

In another alternative embodiment (not shown), a piston spring of the type depicted in FIG. 7 is provided to assist with the displacement of said piston, said link, said tongue receptacle and the user's tongue.

With reference to FIG. 9, in a sixth alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Cylinder 83 is made integral with said shield and is closed at its outer end by cap 84 which is preferably permanently bonded into place. Piston 92 is slideably accommodated within said cylinder and link 20 connected to tongue receptacle (not shown) is connected to said piston by suitable threaded connection means 47. In an alternative embodiment (not shown), said link is fixed to said piston by suitable connection means in the form of bayonet-type, quick- connect/disconnect elements. A suitable displacement spring 93 made from a suitable shape memory polymer material or shape memory metal alloy material is provided with shaped ends 94 which are accommodated within complementary recesses 95 formed in the inner surface of shield 1. The central part of said displacement spring is captured between the outer end of link 20 and the inner surface of piston 92, attachment means 47 passing through a suitable aperture (not shown) provided in it. Said shape memory polymer material or shape memory metal alloy material has a transition temperature close to or slightly below normal human body temperature. The undistorted form of said displacement spring (depicted in broken line as 99) is essentially bell-shaped and the force generated by said spring in reverting to its undistorted form is sufficient to outwardly displace said piston, said tongue receptacle and the relaxed tongue of a user against the urging of extension spring 100. Suitable slots 97 are provided in said piston to accommodate said displacement spring in its distorted form. Suitable recesses 96 are provided in the interior of said shield to accommodate said displacement spring in all modes of movement. In the figure, said displacement spring is depicted with two radially arranged parts which are folded in two places in their distorted form to accommodate the rearward displacement of said piston. In an alternative embodiment (not shown), said displacement spring is made with three or four radially arranged parts. In another alternative embodiment (not shown), shaped ends 94 of said displacement spring are captured in complementary recesses 95 by their engaging suitable catches, pins or the like. The outward displacement of piston 92 is limited by its abutting stop ring 101 accommodated in circumferential groove 102 at the base of cylinder 83. Between usages, said shaped ends of said displacement spring are detached from said complementary recesses, link 20 is detached from piston 92, and said displacement spring is removed and placed in a suitable press (not shown) to deflect its radial parts to their distorted form. Said press is then placed in refrigerated means to cool said distorted displacement spring substantially below said transition temperature. Immediately prior to use, said displacement spring is reinstalled in recesses 96 and slots 97 with its shaped ends engaged with complementary recesses 95. Link 20 is reattached to piston 92 to capture the central part of said displacement spring. In use, said tongue receptacle (not shown) is installed in the oral cavity, the user's tongue is captured in the manner described and the device gripped by closing the bite onto bite blocks 27. As the body heat of the user slowly raises the temperature of said displacement spring, it reverts to its undistorted form, thereby displacing said piston outwardly in said cylinder together with said link and said tongue receptacle such that the user's tongue is drawn out to the desired position to alleviate said symptoms.

With reference to FIG. 10, in a seventh alternative embodiment of said device for the alleviation of snoring and sleep apnoea, shield 1 is made from a suitable rigid polymer material and its inner surfaces 2 are shaped to approximate the shape of the area of the face around the mouth. Said shield, in face view, is optionally made round, oval, triangular, rectangular or other suitable shape. Bite blocks 27 are provided on the inner surface of said shield in a position in which they do not impede outward displacement of the user's tongue. Dome 103 is sealingly fixed to said shield by its threaded extension 104 engaging threaded collar 105 of said dome. Circumferential ring 106 of diaphragm 109 is accommodated in circumferential groove 107 formed at the base of threaded collar 105 and is sealingly captured by the inner end of threaded extension 104 abutting it in its installed position. The inner part of said diaphragm is sealingly captured between the inner end of link 20 and diaphragm spring plate 108. In an alternative embodiment, said dome is secured to said shield by bayonet-type means or the like. Diaphragm spring 110 bears against the inner surface of said dome and diaphragm spring plate 108 and acts to urge said diaphragm and link 20 together with a tongue receptacle (not shown) fixed to the free end of said link into the oral cavity of a user. Nipple 111 is provided on the exterior of said dome and communicates with its interior. Actuator piston 114 is slidingly and sealingly supported in the bore of cylinder 113. Plunger 116 is fixed to said actuator piston and extends outwardly through bearing 117 formed in cylinder cap 119 in which it is slidingly supported. Pressure pad 118 is formed on the outer end of said plunger. Said cylinder cap is fixed to the open end of said cylinder by threaded means, bayonet-type means or the like. Piston spring bears against the closed end of said cylinder and piston 114, acting to urge said piston towards cylinder cap 119. The force exerted by said piston spring 115 is sufficient to overcome that of said diaphragm spring while applying a force to draw a user's tongue out to the desired position to alleviate said symptoms. Cartridge 121 is sealingly fixed to socket 120 formed on the closed end of said cylinder by complementary threaded means. Accommodated within said cartridge is sintered body 123 through which only a small flow of air is permitted. In the preferred embodiment, said sintered body is readily interchangeable with others providing a greater or lesser flow of air. Passing more or less coaxially through said sintered body is a duct (not shown) in which is accommodated a small check valve (not shown) permitting a flow of air out from said cylinder. Port 125 is provided in the end of said cylinder connecting the interior of said cartridge with the interior of said cylinder. Nipple 122 is provided on the outer end of said cartridge and communicates with its interior. A light air line (depicted in broken line as 112) made from a suitable flexible, non-kinking material connects nipple 111 to nipple 122. Said air line is sufficiently long to permit said cylinder to be placed in a suitable position in a bed or other place of rest and to easily reach said shield. In the preferred embodiment, said air line is made from a suitable polymer material and has a bore in the range 1.0 to 2.5 millimetres, but other diameters and materials may optionally be employed. In the preferred embodiment, said cylinder is surrounded by a padded cushion (typical outline depicted in broken line as 124) which prevents its causing any discomfort to a user in bed. Suitable apertures are provided in said cushion to accommodate said cartridge and said piston plunger and allow unobstructed movement of said pressure pad.

In operation, with said piston in the position depicted in the figure, said diaphragm will be drawn substantially into the interior of said dome, carrying said link and said tongue receptacle with it. A user places said shield over his or her face and secures it in position by biting upon said bite blocks. With said shield in place, the user extends his or her tongue, pushing it forcefully into said tongue receptacle, thereby expelling air from said receptacle. The user then fully depresses pressure pad 118, causing said piston to be fully displaced in said cylinder against the urging of piston spring 115 and thereby causing air in said cylinder to be displaced out through said coaxial duct and small check valve of said sintered body and via said air line to said dome. The transfer of air to said dome causes said diaphragm to be outwardly displaced, thereby displacing said link and said tongue receptacle into the user's oral cavity and allowing the user's tongue to return to a more or less normally retracted position. During subsequent sleep, piston spring 115 urges said piston towards said cylinder cap, acting to reduce the pressure at said sintered body. Over time, said sintered body permits a small flow of air from said dome, causing said diaphragm, said link, said tongue receptacle and the user's tongue to be drawn outwardly at an almost imperceptible rate until the user's tongue has been displaced to the desired position to alleviate said symptoms. In an alternative embodiment (not shown), link 20 is made either tubular or solid and telescopic with a complementary element fixed to the central, inner surface of dome 103. Sliding co-operation of said telescopic elements serves to maintain alignment of said link during its said displacement. In another alternative embodiment (not shown), piston 14 is progressively displaced during sleep by a small electric motor powered from a battery pack contained within padded cushion 124. Said electric motor turns a threaded rod which screwably engages a nut within said piston. Limit switches are provided to limit the travel of said piston during operation and resetting. A reset switch is provided to return said piston to its start position prior to use of the present invention.

With reference again to FIGS. 3 and 4, in an alternative embodiment (not shown), support arms 41 and arch 45 are made from a suitable shape memory polymer material or shape memory metal alloy material having a transition temperature equal to or slightly less than normal body temperature. Between usages of the present invention, said support arms and said arch are preferably detached, clamped in a jig (not shown) to generate a desired distorted shape and cooled in suitable refrigerated means to a temperature substantially below said transition temperature of said material. Immediately prior to use, said support arms and said arch are reinstalled on said dentition engagement units and said dentition engagement units properly installed in the user's oral cavity such that said magnets of said magnet supporting plate engage said magnets fixed to or implanted in said uvula. As said support arms and said arch are warmed above said transition temperature of their said material, they slowly resume their undistorted shape, forwardly displacing said magnet supporting plate, said magnets and said uvula, thereby ensuring that said uvula offers no impediment to respiratory airflow.

In alternative embodiments (not shown) of said embodiments employing said shape memory material, said components of said shape memory material are coated with a suitable thermal insulating material to delay heating to the transition temperature of said material.

In other alternative embodiments (not shown), of said embodiments employing said shape memory material, said components of said shape memory material are made laminated with major and minor laminations such that, at said transition temperature, said major laminate tends to resume the undistorted shapes of said components while said minor laminate simultaneously tends to impede said undistorted shape resumption. Said arrangement acts to slow the rate of resumption of said components of their undistorted shapes.

In other alternative embodiments (not shown) of said embodiments employing said shape memory material, said components of said shape memory material are made thin and encapsulated in a thicker, stiffly elastic polymer material which resists distortion and permits only slow resumption of said undistorted shapes.

It will be appreciated by persons skilled in the art that said cylinders of the present invention and their said complementary pistons need not be made in a round transverse cross-sectional shape and are optionally made square, rectangular (with or without rounded corners) or oval.

In alternative embodiments (not shown) of the present invention, a said cylinder having a round transverse cross-sectional shape and its said complementary piston are optionally made with complementary splines and grooves to prevent rotational displacement of said piston within said cylinder during attachment of said tongue receptacle.

It will be appreciated by persons skilled in the art that externally projecting parts of the present invention may readily be made more compact and less protrusive beyond the user's face.

The depiction of springs in the figures is intended only to be indicative of position and general arrangement. In practice, springs would be selected for force and rate and may be of different characteristics.

The present invention is simple in function, easy to use, provides minimal user discomfort and sleep inhibition and reliably and automatically provides the correct degree of tongue extension and stabilization. The invention is readily combined with means to control uvula position, providing an additional therapeutic advantage.

1. Apparatus for the alleviation of snoring and sleep apnoea comprising the combination of a shield with an outwardly-projecting cylinder, said combination supported by a subject against the facial surfaces around the mouth by biting upon bite blocks fixed to the inside of said shield, said cylinder containing a piston or diaphragm connected by a link to a tongue receptacle in which the distal end of the tongue of a subject is captured; said piston or diaphragm being outwardly displaceable within said cylinder by spring means, electrically-operated screw means, physically expansive means or shape-memory material means, said displacement means being contained within the structure of said cylinder and incorporating rate regulating provisions; said displacement means acting to draw said tongue receptacle and said tongue outwardly at a slow, controlled rate during sleep-induced relaxation, thereby reducing any tendency towards airway obstruction.

2. (canceled)

3. The apparatus of claim 1 in which said combined shield and cylinder structure are made from a suitable rigid polymer material.

4. The apparatus of claim 1 in which said shield is anatomically shaped.

5. The apparatus of claim 1 in which said tongue receptacle is made with anatomically correct shaping except for a more or less circumferential restriction to promote sealing of the tongue to it.

6. The apparatus of claim 1 in which said tongue receptacle is made detachable from said link, permitting tongue receptacles of different sizes and configurations to be used.

7. The apparatus of claim 6 in which said tongue receptacle is attached to said link by means of a suitable clip, pin or the like, or by bayonet-type connection means.

8. The apparatus of claim 1 in which only said bite blocks, said link and said tongue receptacle extend into the oral cavity.

9. The apparatus of claim 1 in which said bite blocks are made sufficiently thick to ensure that the teeth of a subject are maintained in spaced-apart relationship sufficiently wide to permit partial egress of said tongue receptacle.

10. (canceled)

11. The apparatus of claim 1 in which supporting bars are incorporated into or fixed to said shield, said supporting bars curving backwardly to contact a subject's face, thereby preventing the imposition of excessive loads upon the face, gums and teeth of the subject while sleeping in a prone attitude.

12. (canceled)

13. (canceled)

14. The apparatus of claim 1 in which said tongue receptacle is provided with two inwardly projecting, primary leaves which pass to either side of the lingual septum and are coated on their upper surfaces with a metal such as gold to make electrical contact with the underside of the base of a subject's tongue.

15. The apparatus of claim 1 in which small metallized secondary leaves are provided on said shield to make electrical contact with the inner lip surfaces of a subject.

16. The apparatus of claim 14 in which said metallized surfaces are connected by suitable insulated conductors to a suitable power source, the delivery of electrical stimulus to the underside of the tongue stimulating it towards an extended position and counteracting any tendency for the tongue to relax into a retracted position.

17. The apparatus of claim 16 in which said power source is located remotely from said shield or accommodated within a small compartment formed on the external surface of said cylinder.

18. The apparatus of claim 16 in which the electrical current supplied by said power source is direct current and the polarity of connection is such that stimulation occurs at said primary leaves.

19. The apparatus of claim 18 in which the voltage and current delivered by said power supply is continuous or in pulsed form and adjusted to be just over the stimulus threshold of a subject.

20. The apparatus of claim 1 in which inwardly extending arms are removably fixed to the lower, inner ends of said bite blocks, said arms supporting at their inner ends an arch, fixed to the centre of which is a magnet supporting plate to which are fixed one or more small, powerful magnets.

21. The apparatus of claim 20 in which said arms are made from a light, stiff material and pass inside the dental arch along the lateral edge of the tongue, offering no impediment to closure of the jaws on said bite blocks.

22. The apparatus of claim 20 in which said arch passes up and over the posterior part of the tongue.

23. The apparatus of claim 20 in which said magnets provide an attractive force to small, powerful magnets attached to the soft palate and uvula of a subject, thereby displacing these parts forwardly and minimising any tendency towards upper airway obstruction.

24. The apparatus of claim 20 in which said magnet supporting plate is made from a soft polymer material within which said magnets are embedded.

25. The apparatus of claim 20 in which the cross-sectional shape of said arms is rectangular or more or less elliptical with the major axis disposed vertically and a ratio of minor to major axes in the range 1:3 to 1:8.

26. The apparatus of claim 20 in which said arms are varied in length, varied in spacing, splayed inwardly or outwardly, angled upwardly or downwardly, or joggled upwardly or downwardly at some point along their length.

27. The apparatus of claim 20 in which said arch is made to extend to a greater or lesser height above the inner ends of said arms, or the plane to which said arch generally conforms is tilted forwardly or rearwardly in relation to the coronal plane.

28. The apparatus of claim 20 in which said arms are made detachable from said bite blocks.

29. The apparatus of claim 1 in which said tongue receptacle is provided with a leaf of thin, stiffly flexible material extending rearwardly from its upper, inner edge, said leaf sitting lightly on the upper surface of the tongue such that the free end of said leaf is positioned adjacent the uvula, magnetic material incorporated into said leaf free end attracting small, powerful magnets attached to the soft palate and uvula of a subject, thereby displacing these parts forwardly and minimising any tendency towards upper airway obstruction.

30. The apparatus of claim 29 in which the inner, free end of said leaf is made from or coated with a magnetic polymer material or has one or more small powerful magnets fixed to it.

31. The apparatus of claim 20 in which said magnets are of the neodymium rare-earth type.

32. The apparatus of claim 1 in which said piston is slidingly accommodated in and sealed to the bore of said cylinder, said piston being initially displaced inwardly by gas pressure injected via a non-return valve and thereafter displaced outwardly by the urging of a spring, the rate of travel of said piston being regulated by the bleeding off of said gas pressure through a small adjustable needle valve, said link, said tongue receptacle and said tongue being displaced outwardly by said piston during sleep-induced relaxation, thereby reducing any tendency towards airway obstruction.

33. The apparatus of claim 1 in which said piston is slidingly accommodated in and sealed to the bore of said cylinder, said piston being displaced outwardly by a depression communicated to said cylinder via a light, flexible air line, said depression being generated by a remote evacuation unit comprising a piston progressively displaced in a cylinder by an electric motor or other form of actuator, said link, said tongue receptacle and said tongue being displaced outwardly by said piston during sleep-induced relaxation, thereby reducing any tendency towards airway obstruction.

34. (canceled)

35. The apparatus of claim 1 in which said piston is slidingly accommodated in the bore of said cylinder and displaced outwardly by thread pads provided on the ends of radially arranged blades fixed to said piston and passing out through slots in said cylinder engaging a complementary threading on the inner surface of a drum surrounding said cylinder, said drum being driven in rotation by a small electric motor, said link, said tongue receptacle and said tongue being displaced outwardly by said piston during sleep-induced relaxation, thereby reducing any tendency towards airway obstruction.

36. The apparatus of claim 35 in which the operation of said electric motor is regulated by software stored in a microprocessor-based control unit.

37. The apparatus of claim 35 in which said electric motor is replaced with a clockwork actuation unit.

38. The apparatus of claim 1 in which said cylinder is made with a large, centrally-located bore in which a principal piston is slidingly accommodated, said centrally-located bore being surrounded by two or more equally-spaced smaller bores, narrow, radially-arranged blades fixed to said principal piston passing out through slots in the wall of said centrally-located bore to support secondary pistons slidingly accommodated in said smaller bores, expansion capsules housed in chambers formed in said shield and coincident with said smaller bores expanding to displace said secondary pistons in their bores together with said principal piston in said centrally-located bore, said link, said tongue receptacle and said tongue being displaced outwardly by said principal piston during sleep-induced relaxation, thereby reducing any tendency towards airway obstruction.

39. The apparatus of claim 38 in which said expansion capsules are made from a finely foamed, stiff elastomeric material which is compressed into compact form in a press and cooled to a temperature in the range −5° C. to −10° C., said expansion capsules slowly resuming their uncompressed shape as they are warmed by body heat.

40. The apparatus of claim 1 in which said piston is slidingly accommodated in the bore of said cylinder and displaced outwardly by a spring against a capsule comprising an elongated, thin, closed tube of an elastomeric material filled with ice or a gel, the rate of displacement of said piston being regulated by progressive melting by body heat of said ice or gel, said link, said tongue receptacle and said tongue being displaced outwardly by said piston during sleep-induced relaxation, thereby reducing any tendency towards airway obstruction.

41. The apparatus of claim 1 in which said piston is slidingly accommodated in the bore of said cylinder and displaced outwardly by a clock spring-type element made from a shape-memory material which progressively resumes a compact shape as it is warmed by body heat, said link, said tongue receptacle and said tongue being displaced outwardly by said piston during sleep-induced relaxation, thereby reducing any tendency towards airway obstruction.

42. The apparatus of claim 41 in which said shape-memory material has a transition temperature equal to or slightly less than normal body temperature.

43. The apparatus of claim 41 in which, before use, said element of shape-memory material is fully extended in a suitable press and cooled to a temperature well below said transition temperature.

44. The apparatus of claim 1 in which said piston is slidingly accommodated in the bore of said cylinder and displaced outwardly by a collapsed bowspring-type element made from a shape-memory material which progressively resumes a bow shape as it is warmed by body heat, said link, said tongue receptacle and said tongue being displaced outwardly by said piston during sleep-induced relaxation, thereby reducing any tendency towards airway obstruction.

45. The apparatus of claim 44 in which slots are provided in said piston to accommodate part of said collapsed bowspring-type element; before use, said bowspring-type element being distorted into its collapsed form in a press and cooled to a temperature well below the transition temperature of said shape-memory material.

46. The apparatus of claim 1 in which the open inner end of a cylinder formed on said shield is closed by a flexible diaphragm which is urged inwardly by a spring; in operation said diaphragm being displaced outwardly against the urging of said spring by a depression communicated to said cylinder via a light, flexible air line, said depression being generated by a remote evacuation unit comprising an actuator piston progressively displaced in an actuator cylinder by the urging of a spring, the rate of displacement of said actuator piston and therefore of said diaphragm being regulated; a plunger being employed to manually depress said actuator piston against the urging of said spring and a check valve permitting reverse air flow past said sintered body, said link, said tongue receptacle and said tongue being displaced outwardly by said diaphragm during sleep-induced relaxation, thereby reducing any tendency towards airway obstruction.

47. The apparatus of claim 46 in which said evacuation unit is surrounded by a padded cushion.

48. The apparatus of claim 46 in which said piston of said evacuation unit is displaced by a nut and screw arrangement driven by a small electric motor.

49. The apparatus of claim 20 in which said inwardly extending arms and said arch are made from a shape-memory material having a transition temperature equal to or slightly less than normal body temperature such that, when warmed by body heat, said arms and said arch resuming their undistorted shapes, thereby forwardly displacing said arch.

50. The apparatus of claim 49 in which, prior to their use, said arms and said arch are detached from said bite blocks, clamped in a jig to distort them and cooled to a temperature substantially below the transition temperature of the material.

51. The apparatus of claim 49 in which said shape-memory material is a metal alloy or polymer material.

52. The apparatus of claim 49 in which said arms and said arch are coated with a thermal insulation material to delay their heating to the transition temperature of the material.

53. The apparatus of claim 49 in which said arms and said arch are made in laminated form with major laminates of a shape-memory material and minor laminates of non shape-memory material, the combination acting to slow the resumption by said components of their undistorted shapes when heated to or above the transition temperature of said shape-memory material.

54. The apparatus of claim 49 in which said arms and said arch are made from a thin shape-memory material and encapsulated in a thicker, stiffly elastic, non shape-memory material, the combination acting to slow the resumption by said components of their undistorted shapes when heated to or above the transition temperature of the shape-memory material.

55. The apparatus of claim 1 in which said link is attached to said piston by a quick-release, bayonet-type connection.

56. The apparatus of claim 15 in which said metallized surfaces are connected by suitable insulated conductors to a suitable power source, the delivery of electrical stimulus to the underside of the tongue stimulating it towards an extended position and counteracting any tendency for the tongue to relax into a retracted position.

57. The apparatus of claim 23 in which said magnets are of the neodymium rare-earth type.

58. The apparatus of claim 29 in which said magnets are of the neodymium rare-earth type.

59. The apparatus of claim 30 in which said magnets are of the neodymium rare-earth type.
